(19) United States
(12) Patent Application Publication
(10) Pub. No.: US 2013/0142761 A1
(43) Pub. Date: Jun. 6, 2013

(54) Method of Enhancing Hematopoietic Cell Transplantation

(71) Applicant: David R. Kaplan, Shaker Heights, OH (US)

(72) Inventor: David R. Kaplan, Shaker Heights, OH (US)

(73) Assignee: Verve, Inc., Pepper Pike, OH (US)

(21) Appl. No.: 13/490,000

(22) Filed: Jun. 6, 2012

Related U.S. Application Data
(60) Provisional application No. 61/494,451, filed on Jun. 8, 2011.

(51) Int. Cl. A61K 35/14

(52) U.S. Cl. CPC A61K 35/14
USPC 424/93.7; 435/7.1; 514/1.1; 435/325; 435/7.92; 435/6.12; 435/7.21

(57) ABSTRACT

The invention relates to a method for enhancing the transplantation of hematopoietic cells to supplement or fully reconstitute the hematopoietic system. The method involves administering cells expressing CD34 and co-expressing HoxB4, wherein the HoxB4 is expressed at levels that provide therapeutically effective amounts of self-renewal of the administered cells. The method also involves administering cells expressing CD34 but not co-expressing HoxB4 or co-expressing HoxB4 in amounts so as to provide therapeutically effective amounts of differentiation of the administered cells into the various progeny cells of the hematopoietic system. To provide such cells to a subject, the invention relates to detecting such cells prior to treatment to ascertain whether such cells are present in clinically relevant amounts.

### FIELD OF THE INVENTION

The invention relates to a method for enhancing the transplantation of hematopoietic cells to supplement or fully reconstitute the hematopoietic system, such as in myeloablated patients or patients otherwise deficient in hematopoietic cells. The method involves administering cells expressing CD34 and co-expressing HoxB4, wherein the HoxB4 is expressed at levels that provide therapeutically effective amounts of self-renewal of the administered cells. The method also involves administering cells expressing CD34 but not co-expressing HoxB4 or co-expressing HoxB4 in amounts so as to provide therapeutically effective amounts of differentiation of the administered cells into the various progeny cells of the hematopoietic system. The HoxB4 is expressed in a range rather than all or nothing expression such as cells co-expressing CD34 and HoxB4, the HoxB4 being expressed in effective amounts for self-renewal or differentiation. To provide such cells to a subject, the invention relates to detecting such cells prior to treatment to ascertain whether such cells are present in clinically relevant amounts. It may also relate to treating a subject so as to provide clinically relevant numbers of such cells, as with specific mobilization agents.

### BACKGROUND OF THE INVENTION

The hematopoietic system can be reconstituted by cells that are the progenitor/stem cells for all blood cells. These stem/progenitor cells can be designated, as in this application, “hematopoietic-reconstituting cells” or “HRC.” Hematopoietic-reconstituting cells are capable of self-renewal and of differentiating into any cell in the hematopoietic system, including lymphocytes, platelets, erythrocytes and myeloid cells. Hematopoietic-reconstituting cells have therapeutic potential as a result of their capacity to restore blood and immune cell function.

Transplantation of CD34⁺ hematopoietic-reconstituting cells is an important treatment modality for malignant and nonmalignant disorders. Most commonly, hematopoietic-reconstituting cells from bone marrow are mobilized into the peripheral blood by pharmacological treatment, thereby facilitating collection. The number of CD34⁺ cells in mobilized blood samples is used to indicate the appropriateness of transplantation although it does not distinguish between the two necessary functions: long-term reconstitution mediated by cells with self-renewing proliferation and short-term hematopoietic differentiation mediated by progenitor cells.

Transplantation of hematopoietic-reconstituting cells from bone marrow, mobilized peripheral blood and umbilical cord blood has been used to treat hematopoietic cancers such as leukemia and lymphomas, and to aid hematopoietic system recovery from high-dose chemotherapy. Myelosuppression and myeloablation often result from high-dose chemotherapy. Prior to treatment with high-dose chemotherapy, bone marrow hematopoietic progenitor/stem cells can be mobilized into the peripheral blood so that peripheral blood can be harvested and stored for later use as a source of hematopoietic-reconstituting cells. The transplantation of the stored hematopoietic-reconstituting cells can rescue hematopoietic functions after high-dose chemotherapy. Allogeneic or autologous hematopoietic-reconstituting cells can be used to mediate hematopoietic reconstitution.

It would be desirable if hematopoietic-reconstituting cells could be definitively identified in a heterogeneous mixture of cells by assessing the cells for expression of at least one marker associated with hematopoietic-reconstituting cells. The CD34⁺ cell number has been used as a marker for the progenitor/stem cell quantity. However, the CD34 molecule is not associated with the two critical hematopoietic-reconstituting cell functions: the capacity for self-renewing proliferation and short-term differentiation into hematopoietic cells. Although the number of CD34⁺ cells can be determined, there remains a large variability in predicting hematopoietic reconstitution.

### SUMMARY OF THE INVENTION

The inventors have discovered that one can predict these two functions in a sample of CD34⁺ cells by assessing the level of HoxB4 co-expression. The inventors assessed the expression levels of HoxB4 in CD34⁺ cells from uniformly mobilized multiple myeloma patients. The expression data were correlated with functional short-term assays for colony formation. The inventors found that colony-forming units were significantly correlated with HoxB4 expression which was explained by CD34⁺ cell numbers. At the same time, analysis of colony-forming units normalized to the CD34⁺ cell count revealed a significant negative correlation with HoxB4 expression.

Thus, the inventors discovered that increased HoxB4 expression enhances CD34⁺ cell number via self-renewing expansion while depreciating the capacity for short-term differentiation per cell. Accordingly, the expression level of HoxB4 in CD34⁺ cells can be used as a measure of whether a sample is appropriate for transplantation. Furthermore, based on these findings CD34⁺ cells in a subject can be manipulated by the addition of specific mobilization agents that increase or decrease HoxB4 expression in CD34⁺ cells. Thus, therapeutically effective amounts of cells co-expressing CD34 at an appropriately therapeutic range of HoxB4 can be obtained and administered to a subject to improve or completely reconstitute the hematopoietic system.

### BRIEF DESCRIPTION OF THE DRAWINGS

FIG. 1a shows representative results for the detection of HoxB4 in hematopoietic-reconstituting cells. Mononuclear cells from 4 different mobilized blood samples were stained for CD34 expression (upper row). The cells were also stained with control immunoglobulin (light outline; lower row) or with specific antibodies (dark outline; lower row) and processed by EAS® for high resolution immunophenotyping. EAS® is an amplification technology disclosed in, for example U.S. Pat. Nos. 6,280,961, 6,335,173, and 6,828,109. The amplified signals (lower row) are shown only for the CD34⁺ events (upper row). Representative results are shown from 4 different donors in order to demonstrate the consistency in CD34⁺ cell delineation.

FIG. 1b shows correlation of HoxB4 expression levels with CFU-GM and log(BFU-E). CD34⁺ cells from various samples of mobilized blood from patients with multiple myeloma were stained for the expression of HoxB4 and the cells were processed by EAS®) for high-resolution immunophenotyping. Each sample was also tested for CFU-GM and BFU-E. The distributions of CFU-GM (upper) and log(BFU-E) (lower) versus HoxB4 median fluorescence ratios are shown for the various samples. Coefficients of correlation and p values are shown.

FIG. 1c shows correlation of HoxB4 expression levels with CFU-GM and log(BFU-E) normalized for CD34+ cell numbers. CD34⁺ cells from various samples of mobilized blood from patients with multiple myeloma were stained for the expression of HoxB4 and the cells were processed by EAS® for high-resolution immunophenotyping. Each sample was also tested for CFU-GM and BFU-E and normalized for the number of CD34+ cells included in the colony forming unit assays. Coefficients of correlation and p values are shown.

FIG. 2 is a flow chart illustrating a method for preparing a subject for donating blood in accordance with an embodiment of the present invention.

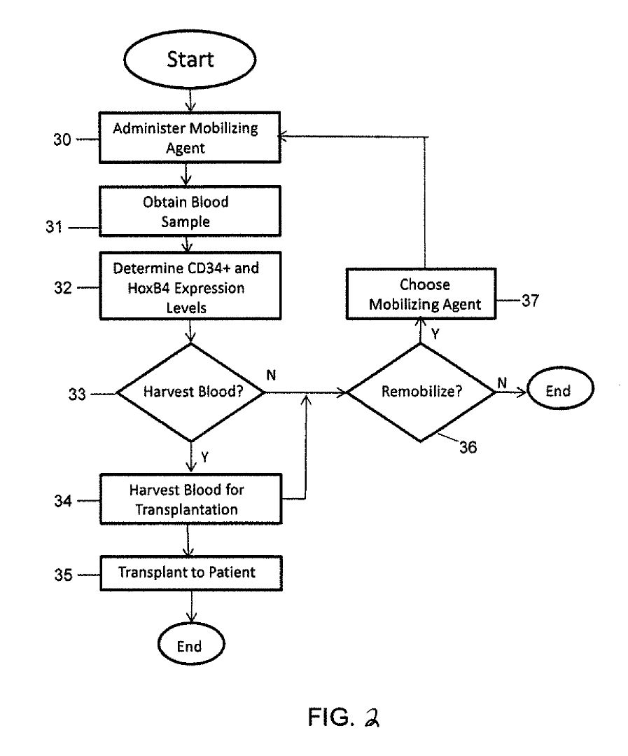

FIG. 3 is a schematic of hematopoietic differentiation.

### DEFINITIONS

“A” or “an” means herein one or more than one; at least one. Where the plural form is used herein, it generally includes the singular.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced potency to achieve the effects described in this application. Following release from storage, and prior to administration to the subject, it may be preferable to again assay the cells for potency. This can be done using any of the assays, direct or indirect, described in this application or otherwise known in the art. Then cells having the desired potency can then be administered to the subject for treatment. Banks can be made using cells derived from the individual to be treated (from their pre-natal tissues such as placenta, umbilical cord blood, or umbilical cord matrix or expanded from the individual at any time after birth). Or banks can contain cells for allogeneic uses.

“Co-administer” means to administer in conjunction with one another, together, coordinately, including simultaneous or sequential administration of two or more agents. In the context of the invention, the two types of CD34⁺ cells can be administered with these alternative regimens.

“Comprised of” is a synonym of “comprising”.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning.

“Decrease” or “reduce” means to prevent entirely as well as to lower.

“Effective amount” generally means an amount which provides the desired effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.” In the context of the invention, effective amounts of the two cell types are those that provide clinically significant self-renewal and differentiation. “Effective amounts of HoxB4 expression” refers to those levels (i.e., ranges) that provide for that clinically-significant self-renewal and differentiation.

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

The term “hematopoietic-reconstituting cell” or “HRC”, as used herein, refers to a progenitor and/or stem cell that can reconstitute all of the hematopoietic cells in a subject. These include, but are not limited to, lymphocytes, platelets, erythrocytes and myeloid cells, including, T cells, B cells (plasma cells), natural killer cells, dendritic cells, monocytes (macrophages), neutrophils, eosinophils, basophils (mast cells), megakaryocytes (platelets), and erythroblasts (erythrocytes). These cells are also capable, in addition to differentiation, of self-renewal, so as to proliferate the stem-progenitor population that is capable of differentiation. Thus, the hematopoietic reconstituting cell may not always be the same cell. But, as shown in this application, the two functions, which are both present in CD34⁺ cells, are differentially effected by the level of expression of HoxB4 in individual CD34⁺ cells.

The term “hematopoietic-reconstituting cell” or “HRC” generally refers to the functions of the cells that provide their ability to reconstitute the hematopoietic system to provide a clinically relevant effect. Technically, the function can be broken down into two functions that are represented by two sets of cells. Accordingly, self-renewing hematopoietic-reconstituting cells are those CD34⁺ cells that express levels of HoxB4 that result in a self-renewal capability of the administered cells. Differentiating hematopoietic-reconstituting cells are a different set of cells, namely those CD34⁺ cells that express HoxB4 at levels that result in differentiation of the administered cells into hematopoietic cell progeny to provide a clinically relevant result. Accordingly when the term “the two CD34⁺ cell types” is used herein, the inventors intend to refer to these two different types of CD34⁺ cells.

The term “HoxB4” is understood to refer to a transcription factor encoded by a gene having, in humans, the sequence shown in, for example, Acampora, D. et al., Nucl. Acids. Res. 17: 10385-10402 (1989). Also see NCBI Reference, Sequence: NM_{—}204015.4, incorporated by reference for the sequence. However, this gene is also known, like most other genes, to contain polymorphisms that still allow the gene to maintain the capacity for HoxB4 function. With respect to this application, it would be sufficient function so as to obtain self-renewal of hematopoietic stem/progenitor cells. The gene also includes, for non-human uses, such as veterinary uses, HoxB4 orthologs from other mammals. These include companion animals, farm animals and sport animals, for example, felines, canines, bovines, equines, porcines, ovines, etc.

Whereas the exemplified hematopoietic-reconstituting cells in this application express HoxB4 naturally (i.e., not by recombinant means such as by exogenous promoter/enhancer insertion into the gene for endogenous HoxB4 or by addition of exogenous HoxB4 coding sequences), the invention could cover cells that are genetically engineered to express desired levels of HoxB4 (for example by increasing the copy number, reducing the copy number, increasing transcription/translation, or decreasing HoxB4 expression, such as by negative regulators such as small molecules, anti-sense RNA and the like).

Use of the term “includes” is not intended to be limiting.

“Increase” or “increasing” means to induce entirely, where there was no pre-existing effect, as well as to increase the degree.

The term “isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only hematopoietic-reconstituting cells. Rather, the term “isolated” indicates that the cells are removed from their natural tissue environment and are present at a higher concentration as compared to the normal tissue environment. Accordingly, an “isolated” cell population may further include cell types in addition to hematopoietic-reconstituting cells and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (for example bone marrow, peripheral blood, umbilical cord blood, etc.).

“Pharmaceutically-acceptable carrier” is any pharmaceutically-acceptable medium for the cells used in the present invention. Such a medium may retain isotonicity, cell metabolism, pH, and the like. It is compatible with administration to a subject in vivo, and can be used, therefore, for cell delivery and treatment.

The term “potency” refers to the ability of a cell population to provide hematopoietic-reconstituting cell effects, i.e., self-renewal and/or differentiation sufficient to achieve a clinically detectable result.

The term “reconstitute” implies a range of increase from a fully or partially deficient hematopoietic system. It is not limited to, for example, cases in which the entire hematopoietic system is ablated. Reduced intensity conditioning is used in HRC transplantation. Reduced intensity conditioning does not result in myeloablation and it is used in patients that are older, in patients who are in complete remission, and in patients with acquired aplastic anemia.

The term “reduce” as used herein means to prevent as well as decrease. In the context of treatment, to “reduce” is to both prevent or ameliorate one or more clinical symptoms. A clinical symptom is one (or more) that has or will have, if left untreated, a negative impact on the quality of life (health) of the subject. This also applies to the biological effects such as self-renewal and differentiation.

“Selecting” a cell with a desired level of potency can mean identifying (as by assay), isolating, and expanding a cell. This could create a population that has a higher potency than the parent cell population from which the cell was isolated.

To select a cell would include both an assay to determine if there is the desired effect and would also include obtaining that cell. The cell may naturally have the effect in that the cell was not incubated with or exposed to an agent that induces the effect. The cell may not be known to have the effect prior to conducting the assay. As the effects could depend on gene expression and/or secretion, one could also select on the basis of one or more of the genes that cause the effects.

Selection could be from cells in a tissue. For example, in this case, cells would be isolated from a desired tissue, expanded in culture, selected for a desired effect, and the selected cells further expanded.

Selection could also be from cells ex vivo, such as cells in culture. In this case, one or more of the cells in culture would be assayed for the effect and the cells obtained that have the effect could be further expanded.

Cells could also be selected for enhanced effect. In this case, the cell population from which the enhanced cell is obtained already has the effect. Enhanced effectiveness means a higher average amount of the effect per cell than in the parent population.

The parent population from which the enhanced cell is selected may be substantially homogeneous (the same cell type). One way to obtain such an enhanced cell from this population is to create single cells or cell pools and assay those cells or cell pools for the effect to obtain clones that naturally have the effect (as opposed to treating the cells with a modulator of the effect) and then expanding those cells that are naturally enhanced.

However, cells may be treated with one or more agents that will enhance the effect of endogenous cellular pathways. Thus, substantially homogeneous populations may be treated to enhance modulation.

If the population is not substantially homogeneous, then, it is preferable that the parental cell population to be treated contains at least 100 of the effective cell type in which enhanced effect is sought, more preferably at least 1,000 of the cells, and still more preferably, at least 10,000 of the cells. Following treatment, this sub-population can be recovered from the heterogeneous population by known cell selection techniques and further expanded if desired.

Thus, desired levels of the effect may be those that are higher than the levels in a given preceding population. For example, cells that are put into primary culture from a tissue and expanded and isolated by culture conditions that are not specifically designed to have the effect, may provide a parent population. Such a parent population can be treated to enhance the average effect per cell or screened for a cell or cells within the population that express higher effect. Such cells can be expanded then to provide a population with a higher (desired) effect.

“Self-renewal” refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. In the context of the present invention, differentiation is into hematopoietic progeny, such as shown in FIG. 4.

“Subject” means a vertebrate, such as a mammal, such as a human. Mammals include, but are not limited to, humans, dogs, cats, horses, cows, and pigs.

The term “therapeutically effective amount” refers to the amount of an agent determined to produce any therapeutic response in a mammal. For example, effective amounts of hematopoietic-reconstituting cells may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms of hematopoietic deficiency.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

“Validate” means to confirm. In the context of the invention, one confirms that a cell is an expressor with a desired potency. This is so that one can then use that cell (in treatment, banking, drug screening, etc.) with a reasonable expectation of efficacy. Accordingly, to validate means to confirm that the cells, having been originally found to have/established as having the desired activity, in fact, retain that activity. Thus, validation is a verification event in a two-event process involving the original determination and the follow-up determination. The second event is referred to herein as “validation.”

### DETAILED DESCRIPTION OF THE INVENTION

The cells of the invention can be used to treat various cancers and immune system disorders, including acute myeloid leukemia, chronic myeloid leukemia, acute lymphocytic leukemia, childhood leukemias, myelodysplastic syndromes, multiple myeloma, lymphoma, chronic lymphocytic leukemia, solid tumors in children, breast cancer, solid tumor in adults, germ cell tumors, primary immunodeficiency diseases, Fanconi anemia, acquired aplastic anemia, acquired immunodeficiency diseases, thalessemia, sickle cell anemia, lysosomal storage disorders and autoimmune diseases. This treatment is also used for multiple sclerosis, systemic sclerosis, rheumatoid arthritis, juvenile idiopathic arthritis, systemic lupus erythematosis, and Crohn's disease, which are all included under the autoimmune disease heading. Additionally, HRC transplantation (autologous) is used in the treatment of cardiovascular disease and stroke.

A distribution of hematopoietic-reconstituting cells that will most likely be effective for long-term reconstitution, but not for short-term differentiation, will have only cells that express a higher range of HoxB4. Long term reconstitution requires self-renewing proliferation. A distribution of hematopoietic-reconstituting cells that will most likely be effective for short-term differentiation, but not for long-term reconstitution, will have only cells that express a lower range of HoxB4.

### Embodiments of the Invention

In one embodiment the invention is directed to a method for assessing the capacity of a sample to therapeutically effect hematopoietic reconstitution in a subject, the method comprising: assessing co-expression of CD34 and HoxB4 in individual cells in the sample and determining the number of cells co-expressing CD34 and levels of HoxB4 that effect therapeutic levels of self-renewing proliferation and the number of cells co-expressing CD34 and levels of HoxB4 that effect therapeutic levels of differentiation into cells of the hematopoietic lineage.

In one embodiment the invention is directed to a method to therapeutically effect hematopoietic reconstitution in a subject, the method comprising administering to a subject an agent that increases the expression of HoxB4 in CD34⁺ cells in the subject so as to provide a therapeutically effective amount of cells co-expressing CD34 and levels of HoxB4 that effect therapeutic levels of self renewing proliferation.

In one embodiment the invention is directed to a method to therapeutically effect hematopoietic reconstitution in a subject, the method comprising administering an agent that decreases the levels of HoxB4 in CD34⁺ cells in a subject so as to produce a therapeutically effective amount of cells co-expressing CD34 and levels of HoxB4 that effect therapeutic levels of differentiation into hematopoietic cells.

In one embodiment the invention is directed to the methods wherein the sample is obtained from bone marrow.

In one embodiment the invention is directed to the above methods wherein the sample is obtained from blood.

In one embodiment the invention is directed to the methods wherein the blood is umbilical cord blood.

In one embodiment the invention is directed to the methods wherein the blood is mobilized peripheral blood.

In one embodiment the invention is directed to a method to prepare a subject to donate blood for hematopoietic-reconstituting cell transplantation, the method comprising obtaining a blood sample containing hematopoietic cells from the subject; determining the CD34 and HoxB4 expression levels in individual cells from the blood sample; and administering to the subject a mobilizing agent if it is determined that the blood sample does not contain a therapeutically effective amount of either (a) CD34⁺ cells expressing levels of HoxB4 for self-renewal of hematopoietic-reconstituting cells or (b) CD34⁺ cells expressing levels of HoxB4 for differentiation of hematopoietic-reconstituting cells into hematopoietic cells.

In one embodiment the invention is directed to the above methods wherein the sample is obtained from bone marrow.

In one embodiment the invention is directed to the above methods wherein the blood sample is obtained from mobilized peripheral blood.

In one embodiment the invention is directed to the above methods wherein the blood sample is obtained from umbilical cord blood.

In one embodiment the invention is directed to the above methods comprising the step of administering a mobilizing agent to the subject prior to the step of obtaining a blood sample.

In one embodiment the invention is directed to a method for transplanting hematopoietic-reconstituting cells in a subject in need thereof, the method comprising administering to the subject nucleated blood cells comprising a therapeutically effective amount of (a) CD34⁺ cells expressing levels of HoxB4 for self-renewal of hematopoietic-reconstituting cells and (b) CD34⁺ cells expressing levels of HoxB4 for differentiation of hematopoietic-reconstituting cells into hematopoietic cells.

In one embodiment the invention is directed to the above methods wherein the subject has undergone myeloablation.

In one embodiment the invention is directed to the above methods wherein assessing the co-expression of CD34⁺ and HoxB4 on individual cells is performed by flow cytometry. In one embodiment the invention is directed to the above methods wherein the two types of CD34⁺ cells expressing the desired levels of HoxB4 are isolated.

In one embodiment the invention is directed to the above methods wherein the subject has a disorder treatable by hematopoietic stem cell transplantation.

In one embodiment the invention is directed to the above methods wherein the disorder is a hematopoietic deficiency or malignancy.

In one embodiment the invention is directed to the above methods wherein the CD34⁺ cells expressing the two levels of HoxB4 are in combination before administration to the subject.

In one embodiment the invention is directed to the above methods wherein the CD34⁺ cells expressing the two levels of HoxB4 are administered sequentially.

In one embodiment, transplantation is with autologous hematopoietic-reconstituting cells.

In another embodiment, transplantation is with allogenic hematopoietic-reconstituting cells.

The two types of CD34⁺ cells may be combined before administration to the subject. In another aspect the two types of CD34⁺ cells are administered separately, including sequentially (in either order).

Various techniques for assessing HoxB4 expression in CD34⁺ cells that may be used include, but are not limited to, flow cytometry, flow cytometry with tyramide deposition technology (EAS®), single cell mass cytometry, immunohistochemistry, western analysis after CD34⁺ cell isolation, enzyme-linked immunosorbent assays (ELISA), and nucleic acid analysis including single cell polymerase chain reaction (PCR).

In one embodiment, the levels of gene expression are assessed by EAS®, disclosed, for example, in U.S. Pat. Nos. 6,280,961, 6,335,173, and 6,828,109, incorporated by reference for the amplification methods disclosed.

The CD34⁺ cells may be obtained from bone marrow, umbilical cord blood or peripheral blood. In peripheral blood, CD34⁺ cells occur naturally and can be mobilized from the bone marrow by pharmacological treatment.

In one embodiment, a mobilizing agent is administered to the subject if it is determined that the blood sample does not contain sufficient hematopoietic-reconstituting cells with levels of HoxB4 effective for clinically relevant self-renewal and/or differentiation. In another embodiment, the mobilizing agent is administered prior to assessing the level of the hematopoietic reconstituting cells. In other embodiments, the process is iterative with assessment followed by mobilization and further assessments/mobilizations depending upon the results with the mobilizing agent.

The mobilizing agent may increase the number of hematopoietic reconstituting cells from around 2×-2,000× or more. Ranges can be around 2×-10×, 10×-50×, 50×-100×, 100×-500×, 500×-1000×, 1000×-1500×, and 1500×-2000×. In the case of modulators of HoxB4, similarly, an increase of HoxB4 levels could be in the same ranges as well as a decrease in the same ranges.

Different agents may be used for mobilizing hematopoietic-reconstituting cells, depending on the types of blood cell and/or expression levels desired. In addition, the timing of the collection of the blood sample may affect the types of cells and/or expression levels of the cells collected. For example, it may be possible that early in a mobilization, a predominance of the CD34⁺ cells that express levels of HoxB4 for differentiation, and later in the mobilization, a predominance of the CD34⁺ cells that express levels of HoxB4 for self-renewal.

Various compounds are known that modulate HoxB4 expression. These are available in the literature and include, but are not limited to, Seet L F, et al., (2009) Eur. J. Haemotol. 82:124-132; Giannola D M, et al., (2000) J. Exp. Med. 192: 1479-1490; Tang Y, et al., (2009) British J. Haemotol. 144:603-612; Schiedlmeier B, et al., (2003) Blood 101:1759-1768; Antonchuk J., et al., (2001) Exp. Hematol. 29:1125-1134; Purton L E, et al., (2006) J. Exp. Med. 203:1283-1293; Kirito K., et al., (2003) Blood 102:3172-3178; Zhong Y., et al., (2010) Biochem. Biophys. Res. Com. 398:377-382, all incorporated by reference for teaching these compounds.

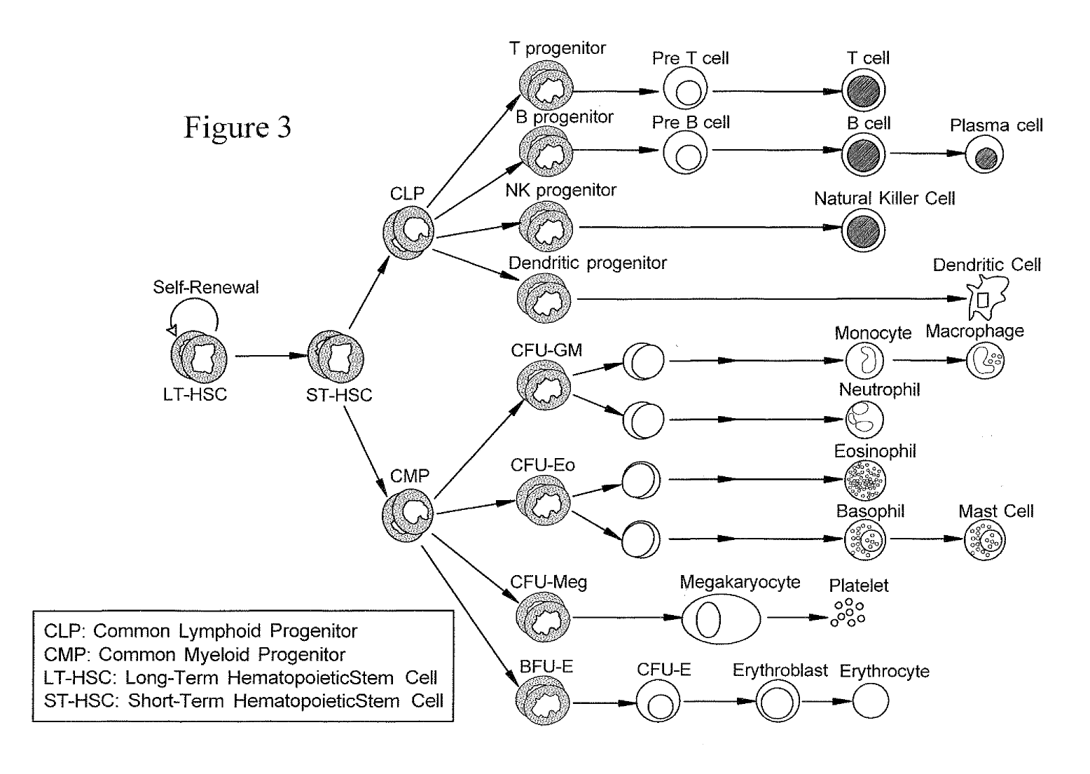

Referring to FIG. 3, a method for preparing a subject for donating blood for hematopoietic reconstitution in accordance with an embodiment of the present invention is illustrated in a flow chart. At step 30, a mobilizing agent is administered to the subject. A blood sample is obtained from the subject at step 31. At step 32, the CD34⁺ and HoxB4 expression levels of the blood sample are determined. A decision is made at step 33 whether the mobilized blood should be collected (i.e., harvested) from the subject based on the results obtained at step 32. If the decision is made to proceed with harvesting, the process continues to step 34 where the blood is collected before transplantation at step 35. The harvested blood may be stored prior to transplantation.

After harvesting the blood at step 33, a decision may be made at step 36 whether to remobilize the subject in order to obtain additional blood from the subject. If the decision is made to proceed with remobilizing the subject at step 36, the process proceeds to step 37 where the mobilizing agent is selected based on the desired characteristics of the additional blood to be drawn. The process then proceeds on to step 30. If the decision is made at step 36 not to remobilize the subject, the process ends.

The method illustrated in FIG. 3 may be modified such that one or more additional blood samples may be obtained from the subject after the initial mobilization has occurred at step 30. The subsequent samples may be obtained at various pre-determined intervals of time after mobilization has occurred because, as described above, the HoxB4 expression levels of the CD34⁺ cells collected may change in the time period following mobilization.

According to the method of the present invention, CD34⁺ cells with the desired HoxB4 expression levels can be obtained from different mobilizations, and then administered to the patient in combination or sequentially.

In one embodiment the hematopoietic reconstituting cells that are administered to the subject are autologous. In another embodiment they are allogeneic.

In a further embodiment, the hematopoietic reconstituting cells that are isolated from a subject for further administration are much more concentrated than they were in vivo. In fact these cells may form a substantially homogeneous population. Accordingly, these cells (both high and low HoxB4 expressers) can be used to directly create a source of cells to be administered at a later date and stored without further manipulation. Alternatively, the cells may be cultured, for example, expanded prior to or after storage. Accordingly, one can create a master cell bank with these cells, aliquots of which can be thawed and used for later administration with or without further expansion.

Because the methods described herein allow the isolation and concentration of the two cell types described herein, the invention is also directed to novel compositions containing these cells at various levels of purity that have not been obtained before. These include about 5-10%, 10-20%, 20-30%, 30-40%, 40-50%, 50-60%, 60-70%, 70-80%, 80-90%, and 90-100%.

### Cell Culture

In general, cells useful for the invention can be maintained and expanded in culture medium that is available and well-known in the art. Also contemplated is supplementation of cell culture medium with mammalian sera. Additional supplements can also be used advantageously to supply the cells with the necessary trace elements for optimal growth and expansion. Hormones can also be advantageously used in cell culture. Lipids and lipid carriers can also be used to supplement cell culture media, depending on the type of cell and the fate of the differentiated cell. Also contemplated is the use of feeder cell layers.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. One embodiment of the present invention utilizes fibronectin. See, for example, Ohashi et al., Nature Medicine, 13:880-885 (2007); Matsumoto et al., J Bioscience and Bioengineering, 105:350-354 (2008); Kirouac et al., Cell Stem Cell, 3:369-381 (2008); Chua et al., Biomaterials, 26:2537-2547 (2005); Drobinskaya et al., Stem Cells, 26:2245-2256 (2008); Dvir-Ginzberg et al., FASEB J, 22:1440-1449 (2008); Turner et al., J Biomed Mater Res Part B: Appl Biomater, 82B:156-168 (2007); and Miyazawa et al., Journal of Gastroenterology and Hepatology, 22:1959-1964 (2007)).

Once established in culture, cells can be used fresh or frozen and stored as frozen stocks, using, for example, DMEM with 40% FCS and 10% DMSO. Other methods for preparing frozen stocks for cultured cells are also available to those of skill in the art.

### Pharmaceutical Formulations

In certain embodiments, the cell populations are present within a composition adapted for and suitable for delivery, i.e., physiologically compatible.

In some embodiments the purity of the cells for administration to a subject is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

The choice of formulation for administering the cells for a given application will depend on a variety of factors. Prominent among these will be the species of subject, the nature of the condition being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant.

In some embodiments, cells/medium are formulated in a unit dosage injectable form, such as a solution, suspension, or emulsion. Pharmaceutical formulations suitable for injection of cells/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

The dosage of the cells will vary within wide limits and will be fitted to the individual requirements in each particular case. In general, in the case of parenteral administration, it is customary to administer from about 0.01 to about 20 million cells/kg of recipient body weight. The number of cells will vary depending on the weight and condition of the recipient, the number or frequency of administrations, and other variables known to those of skill in the art. The cells can be administered by a route that is suitable for the tissue or organ. For example, they can be administered systemically, i.e., parenterally, by intravenous administration, or can be targeted to a particular tissue or organ; they can be administrated via subcutaneous administration or by administration into specific desired tissues.

The cells can be suspended in an appropriate excipient in a concentration from about 0.01 to about 5×10⁶ cells/ml. Suitable excipients for injection solutions are those that are biologically and physiologically compatible with the cells and with the recipient, such as buffered saline solution or other suitable excipients. The composition for administration can be formulated, produced, and stored according to standard methods complying with proper sterility and stability.

### Dosing

Doses for humans or other mammals can be determined without undue experimentation by the skilled artisan, from this disclosure, the documents cited herein, and the knowledge in the art. The dose of cells/medium appropriate to be used in accordance with various embodiments of the invention will depend on numerous factors. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration.

The optimal dose of cells could be in the range of doses used for autologous, mononuclear bone marrow transplantation. For fairly pure preparations of cells, optimal doses in various embodiments will range from 10⁴ to 10^{g} cells/kg of recipient mass per administration. In some embodiments the optimal dose per administration will be between 10⁵ to 10⁷ cells/kg. In many embodiments the optimal dose per administration will be 5×10⁵ to 5×10⁶ cells/kg. By way of reference, higher doses in the foregoing are analogous to the doses of nucleated cells used in autologous mononuclear bone marrow transplantation. Some of the lower doses are analogous to the number of CD34⁺ cells/kg used in autologous mononuclear bone marrow transplantation.

In various embodiments, cells/medium may be administered in an initial dose, and thereafter maintained by further administration. Cells/medium may be administered by one method initially, and thereafter administered by the same method or one or more different methods. The levels can be maintained by the ongoing administration of the cells/medium. Various embodiments administer the cells/medium either initially or to maintain their level in the subject or both by intravenous injection. In a variety of embodiments, other forms of administration are used, dependent upon the patient's condition and other factors, discussed elsewhere herein.

Cells/medium may be administered in many frequencies over a wide range of times. Generally lengths of treatment will be proportional to the length of the disease process, the effectiveness of the therapies being applied, and the condition and response of the subject being treated.

### Uses

Administering the cells is useful in any number of pathologies, including, but not limited to, those listed herein.

In addition, other uses are provided by knowledge of the biological mechanisms described in this application. One of these includes drug discovery. This aspect involves screening one or more compounds for the ability to affect the cell's ability to achieve any of the effects described in this application. Accordingly, the assay may be designed to be conducted in vivo or in vitro.

Gene expression can be assessed by directly assaying protein or RNA. This can be done through any of the well-known techniques available in the art, such as by PACS and other antibody-based detection methods and PCR and other hybridization-based detection methods. Indirect assays may also be used for expression, such as the effect of gene expression.

Assays for potency may be performed by detecting the genes modulated by the cells Detection may be direct, e.g., via RNA or protein assays or indirect, e.g., biological assays for one or more biological effects of these genes.

A further use for the invention is the establishment of cell banks to provide cells for clinical administration. Generally, a fundamental part of this procedure is to provide cells that have a desired potency for administration in various therapeutic clinical settings.

In a specific embodiment of the invention, the cells are selected for having a desired potency for hematopoietic reconstitution (or the self-renewal or differentiation components).

Any of the same assays useful for drug discovery could also be applied to selecting cells for the bank as well as from the bank for administration.

Accordingly, in a banking procedure, the cells (or medium) would be assayed for the ability to achieve any of the above effects. Then, cells would be selected that have a desired potency for any of the above effects, and these cells would form the basis for creating a cell bank.

It is also contemplated that potency can be increased by treatment with an exogenous compound, such as a compound discovered through screening the cells with large combinatorial libraries. These compound libraries may be libraries of agents that include, but are not limited to, small organic molecules, antisense nucleic acids, siRNA DNA aptamers, peptides, antibodies, non-antibody proteins, cytokines, chemokines, and chemo-attractants. For example, cells may be exposed such agents at any time during the growth and manufacturing procedure. The only requirement is that there be sufficient numbers for the desired assay to be conducted to assess whether or not the agent increases potency. Such an agent, found during the general drug discovery process described above, could more advantageously be applied during the last passage prior to banking.

A further use is to assess the efficacy of the cell to achieve any of the above results as a pre-treatment diagnostic that precedes administering the cells to a subject. Moreover, dosage can depend upon the potency of the cells that are being administered. Accordingly, a pre-treatment diagnostic assay for potency can be useful to determine the dose of the cells initially administered to the patient and, possibly, further administered during treatment based on the real-time assessment of clinical effect.

It is also to be understood that the cells of the invention can be used not only for purposes of treatment, but also research purposes, both in vivo and in vitro to understand the mechanism involved normally and in disease models. In one embodiment, assays, in viva or in vitro, can be done in the presence of agents known to be involved in the biological process. The effect of those agents can then be assessed. These types of assays could also be used to screen for agents that have an effect on the events that are promoted by the cells of the invention. Accordingly, in one embodiment, one could screen for agents in the disease model that reverse the negative effects and/or promote positive effects. Conversely, one could screen for agents that have negative effects in a non-disease model.

### Compositions

The invention is also directed to cell populations with specific potencies for achieving any of the effects described herein. As described above, these populations are established by selecting for cells that have desired potency. These populations are used to make other compositions, for example, a cell bank comprising populations with specific desired potencies and pharmaceutical compositions containing a cell population with a specific desired potency.

### Example

Since CD34 is expressed on hematopoietic-reconstituting cells, flow cytometric-based enumeration of CD34⁺ cells has been used as an assay to assess the capacity of a sample to mediate hematopoietic reconstitution. Nevertheless, CD34 expression is not a functional quality biomarker for hematopoietic-reconstituting cells since a biological role of the CD34 molecule itself has not been assigned to either the capacity to differentiate into cells of the hematopoietic lineage or with the property of self-renewal. The capacity of hematopoietic reconstituting cell preparations to reconstitute the hematopoietic system has been assessed by a granulocyte-monocyte colony-forming unit assay (CFU-GM) and an erythrocytic burst-forming unit assay (BFU-E); however, these assays measure function and do not give any information at a molecular level.

Many molecules expressed in hematopoietic-reconstituting cells have been associated with the potential for differentiation into cells of the various hematopoietic lineages and for the capacity for self-renewal including transcription factors (1-14), pathway molecules (15-21), and surface receptors (22-25). Most of these studies have been performed in murine models with knock-out genetic approaches. It has been difficult to assess the expression of these molecules in human hematopoietic-reconstituting cells because they are expressed at low abundance which precludes quantitative information by standard methods of flow cytometry and because hematopoietic-reconstituting cells are a small subpopulation of the cells collected making it difficult to determine by western analysis.

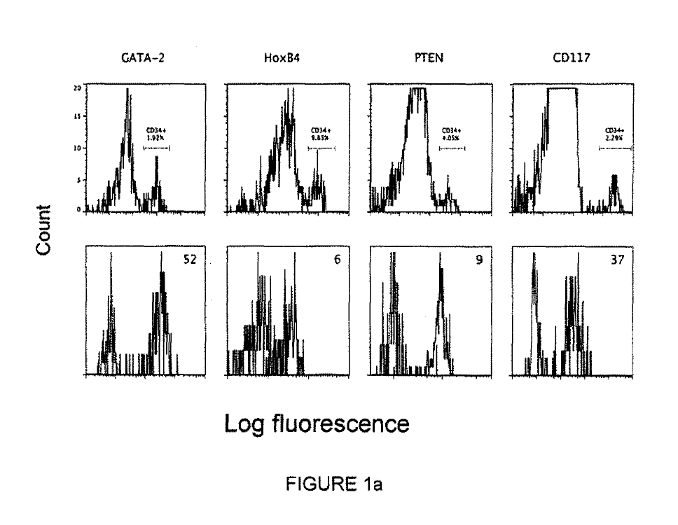

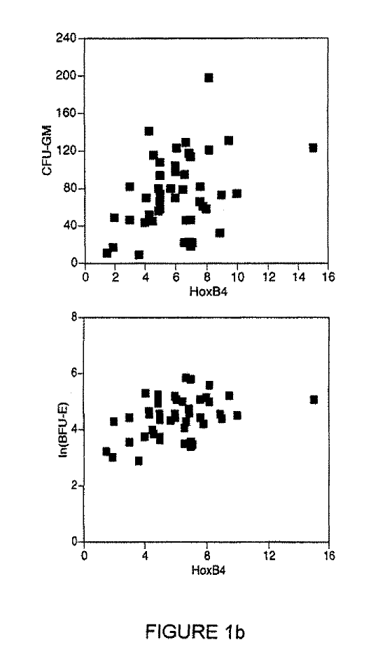

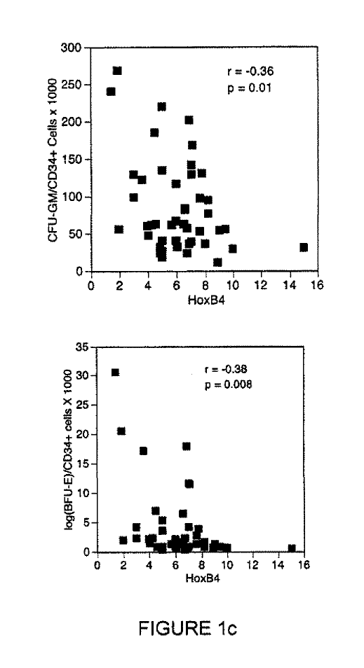

Representative results are shown in FIG. 1a. The CD34⁺ subpopulation of cells was clearly delineated. This allowed a clear assessment of expression in hematopoietic-reconstituting cells. Also, the expression of the various molecules in hematopoietic-reconstituting cells was unimodal and discrete. That indicates that expression levels were precisely and accurately indicated by the calculation of median fluorescence ratios.

Additionally, mobilized blood samples were assayed for CFU-GM and BFU-E. Correlations with the various molecules were sought. Since the distribution of BFU-E was skewed, logarithmic transformation was used for this analysis. The expression level of HoxB4 was the only molecule that showed a statistically significant positive association (FIG. 1b). And it was correlated with both CFU-GM (r=0.423; p=0.003) and logarithm-transformed BFU-E (r=0.355; p=0.014). CFU-GM was also marginally significantly correlated with GATA-2 expression levels (r=0.276; p=0.06).

The proportion of CD34⁺ cells among the nucleated blood cells in the mobilized blood samples demonstrated a highly significant correlation with both. CFU-GM (r=0.734; p<0.0001) and log(BFU-E) (r=0.798; p<0.0001). Moreover, multiple linear regression analysis demonstrated that the proportion of CD34⁺ cells explains the univariate correlation of HoxB4 with the functional assays.

These samples were also assessed for the potential association of the number of colonies normalized to the number of CD34⁺ cells in the culture with HoxB4 expression levels (FIG. 1c). Although a positive correlation between HoxB4 and CFU-GM and between HoxB4 and log (BFU-E) (FIG. 1b) were shown, there were statistically significant negative correlations between HoxB4 expression levels and the number of colonies (both CFU-GM and log(BFU-E)) normalized by the input CD34⁺ cell count.

HoxB4 has been shown to mediate expansion of hematopoietic-reconstituting cells in a variety of settings (1, 32-35). Ectopic expression of the molecule in cord blood cells, embryonic stem cells, and CD34⁺ hematopoietic-reconstituting cells mediates the expansion of these cells in vitro while retaining self-renewal capabilities.

By assessing colony formation with normalization to the input number of CD34⁺ cells, the inventors found that HoxB4 expression is important for the expansion of hematopoietic-reconstituting cells in a way that preserves self-renewal and at the same time it is associated with a decrease in the short-term capacity of the cells to differentiate into the various hematopoietic lineages. Since these functions may be mutually exclusive (42), it is important to ascertain that any inoculum used for hematopoietic reconstitution, such as in myeloablated persons, contain both of these types of hematopoietic-reconstituting cell. New mobilization protocols can be fruitfully devised that focus on these two types of cells either with their presence in the same sample or in two different samples that could be transplanted together. The assessment of HoxB4 expression levels in CD34⁺ hematopoietic-reconstituting cells can be used to ascertain the status of the cells in terms of both self-renewing cellular expansion and short-term hematopoietic differentiation.

### Methods

### Patient Samples

Mobilized blood samples from 52 collections representing 44 unique donors with the diagnosis of multiple myeloma were obtained at University Hospitals Case Medical Center under approval from the Institutional Review Board. Hematopoietic-reconstituting cells were uniformly mobilized from patients with intravenous cyclophosphamide 4 g/m², filgrastim (Amgen, Thousand Oaks, Calif.) 10 meg/kg once or twice daily subcutaneous (determined by resting-state blood CD34⁺ cell concentration), and prednisone 2 mg/kg/day by mouth for 4 days. Apheresis was performed when blood CD34⁺ cell count exceeded 10/meL. Filgrastim was continued until the last day of apheresis. A multi-lumen central venous apheresis catheter was placed either in the internal jugular or subclavian vein for blood cell mobilization and subsequent transplantation. The mononuclear cells were isolated by ficoll/hypaque discontinuous gradient centrifugation and cryopreserved in dimethylsulfoxide for later analysis.

### Antibodies

Monoclonal antibodies specific for CD34 were obtained from BioLegend (San Diego, Calif.). Antibodies specific for HoxB4 were from Epitomics (Burlingame, Calif.).

### Flow Cytometric Analysis

The samples were analyzed for the expression of HoxB4, by Pathfinder Biotech (Cleveland, Ohio) using enzymatic amplification staining (EAS®) as previously described (26-30). EAS® is a validated, catalyzed reporter deposition technology based on the enzymatic activity of peroxidase. The events were gated with the characteristic forward scatter and side scatter for CD34⁺ cells. CD34 counterstaining was included in all samples. In 3 of the 52 samples obtained, no definitive peak of CD34⁺ events could be detected; consequently, these samples were not analyzed further. The median fluorescence ratio for HoxB4 expression was obtained for CD34⁺ events from the median fluorescence intensities of specific antibodies versus matched control immunoglobulin. Multiple quality control features for high-resolution immunophenotyping have been ascertained. Most importantly, analytical reproducibility was demonstrated by staining identical frozen aliquots of Jurkat cells for a variety of intracellular analytes (30). Additionally, carboxylated polystyrene beads substituted with various amounts of human IgG (as an analyte) were used to demonstrate the linearity of detection by EAS® at levels under the level of detection by indirect staining (27). Thus, the data obtained are reproducible and quantitative.

### Colony-Forming Unit Assays

Mononuclear cells (1×10⁵) were grown in duplicate in methylcellulose (Stem Cell Technologies; Vancouver, Canada) containing 10 ng/ml IL-3, 3 U/ml EPO, 50 ng/ml SCF, and 10 ng/ml GM-CSF. Hemin (0.1 mM; Sigma Chemicals; St. Louis, Mo.), and the cells were incubated at 37° C. and 5% CO₂. After 12-14 days, colonies greater than 50 cells were identified by morphology, enumerated and expressed as total CFU-GM and BFU-E (31).

### Statistical Analysis

The association between two continuous measurements was estimated using Pearson correlation coefficient after checking normality assumption. Logarithmic transformation was performed for those measures whose normality is violated. In the model diagnosis, outliers and influential observations were identified using both the studentized residuals and Cook's distance. Statistical analyses were performed using SAS software (Cary, N.C.). All tests were two-sided and p-value less than 0.05 were considered statistically significant.

### REFERENCES

1. Sauvageau G, Thorsteinsdottir U, Eaves C J, Lawrence H J, Largman C, Lansdorp P M, Humphries R K. Overexpression of HOXB4 in hematopoietic cells causes the selective expansion of more primitive populations in vitro and in vivo. Genes & Dev 1995; 9:1753-1765.
2. Unger C, Karner E, Treschow A, Stellan B, Fel'din U, Concha H, Wendel M, Hovatta O, Aints A, Ahrlund-Richter L, Dilber M S. Lentiviral-mediated HoxB4 in human embryonic stem cells initiates early hematopoiesis in a dose-dependent manner but does not promote myeloid differentiation. Stem Cells 2008; 26:2455-2466.
3. Wilson A, Murphy M J, Oskarsson T, Kaloulis K, Betess M D, Oser G M, Pasche A C, Knabenhans C, MacDonald H R, Trumpp A c-Myc controls the balance between hematopoietic stem cell self-renewal and differentiation. Genes & Dev 2004; 18:2747-2763.
4. Baena E, Ortiz M, Martinez-A C, de Alboran I M. c-Myc is essential for hematopoietic stem cell differentiation and regulates Lin-Sca-1+c-Kit-cell generation through p21. Exp Hematol 2007; 35; 1333-1343.
5. Laurenti E, Varnum-Finney B, Wilson A, Ferrero I, Blance-Bose W E, Ehninger A, Knoepfler P S, Cheng P-F, MacDonald H R, Eisenman R N, Bernstein I D, Trumpp A. Hematopoietic stem cell function and survival depend on c-Myc and N-Myc activity. Cell Stem Cell 2008; 3:611-624.
6. Satoh Y, Matsumura I, Tanaka H, Ezoe S, Sugahara H, Mizuki M, Shibayama H, Ishiko E, Ishiko E, Ishiko J, Nakajima K, Kanakura Y. Roles for c-Myc in self-renewal of hematopoietic stem cells. J Biol Chem 2004; 279; 24986-24993.
7. Tsai F-Y, Keller G, Kuo F C, Weiss M, Chen J, Rosenblatt M, Alt F W, Orkin S H. An early haematopoietic defect in mice lacking the transcription factor GATA-2. Nature 1994; 371:221-226.
8. Heyworth C, Gale K, Dexter M, May G, Enver T. A GATA-2/estrogen receptor chimera functions as a ligand-dependent negative regulator of self-renewal. Genes & Dev 1999; 13:1847-1860.
9. Ezoe S, Matsumura I, Nakata S, Gale K, Ishihara K, Minegishi N, Machii T, Kitamura T, Yamamoto M, Enver T, Kanakura Y. GATA-2/estrogen receptor chimera regulates cytokine-dependent growth of hematopoietic cells through accumulation of p21wafl and p27kipl proteins. Blood 2002; 100:3512-3520.
10. Tipping A J, Pina C, Castor A, Hong D, Rodrigues N P, Lazzari L, May G E, Jacobsen S E W, Enver T. High GATA-2 expression inhibts human hematopoietic stem and progenitor cell function by effects on cell cycle. Blood 2009; 113:2661-2672.
11. Park, I., Qian, D., Kiel, M., Becker, M. W., Pihalja, M., Weissman, I. L., Morrison, S I, & Clarke, M. F. Bmi-1 is required for maintenance of adult self-renewing haematopietic stem cells. Nature 423, 302-305 (2003).
12. Rizo, A., Olthof, S., Han, L., Vellenga, E., de Haan, G., & Schuring a, J. J. Repression of BMI1 in normal and lekemic human CD34+ cells impairs self-renewal and induces apoptosis. Blood 114, 1498-1505 (2009).
13. Liakhovitskaia, A., Gribi, R. Stamateris, E., Villain, G., Jaffredo, T., Wilkie, R., Gilchrist, D., Yang, J., Ure, J., & Medvinsky, A. Restoration of Runxl expression in the Tie2 cell compartment rescues definitive haematopoietic stem cells and extends life of Runxl knockout animals until birth. Stem Cells 27, 1616-1624 (2009).
14. Semerad, C. J., Mercer, E. M., Inlay, M. A., Weissman, I. L., & Murre, C. E2A proteins maintain the hematopoietic stem cell pool and promote the maturation of myelolymphoid and myeloerythroid progenitors. Proc. Natl. Acad. Sci. USA 106, 1930-1935 (2009).
15. Zhang J, Grindley J C, Yin T, Jayasinghe S, He X C, Ross J T, Haug J S, Rupp D, Porter-Westpfall K S, Wiedemann L M, Wu H, Li L. PTEN maintains haematopoietic stem cells and acts in lineage choice and leukaemia prevention. Nature 2006; 441:518-522.
16. Juntilla, M. M., Patil, V. D., Calamito, M, Joshi, R. P., Birnbaum, M. J., & Koretzky, G. A. AKT1 and AKT2 maintain hematopietic stem cell function by regulating reactive oxygen species. Blood 115, 4030-4038 (2010).
17. Reya, T, Duncan, A. W., Ailles, L., Domen, J., Scherer, D. C., Willert, K., Hintz, L., Nusse, R., & Weissman, I. L. A role for Wnt signaling in self-renewal of haematopoietic stem cells. Nature 423, 409-414 (2003).
18. Kim, J., Kang, Y., Park, G., Kim, M., Park, Y., Kim, H., Leem, S., Chu, I., Lee, J., Jho, E., & Oh, I. Identification of a stroma-mediated Wnt/β-catenin signal promoting self-renewal of hematopoietic stem cells in the stem cell niche. Stem Cells 27, 1318-1329 (2009).
19. Li, G., Wang, Z., Miskimen, K. L., Zhang, Y., Tse, W., Bunting, K. D. Gab2 promotes hematopoietic stem cell maintenance and self-renewal synergistically with STATS. PLoS One 5(2): e9152. Doi:10.1271/journal.pone.0009152.
20. Gu, H., Pratt, J C, Burakoff, S J, Neel, B G. Cloning of p97/Gab2, the major SHP2-binding protein in hematopoietic cells, reveals a novel pathway for cytokine-induced gene activation. Mal. Cell 2:729-740 (1998).
21. Nishida, K, Yoshida, Y, Itoh, M, Fukada, T, Ohtani, T, Shirogane T, Atsumi T, Takahashi-Tezuka M, Ishihara K, Hibi, M, Hirano, T. Gab-family adapter proteins act downstream of cytokine and growth factor receptors and T- and B-cell antigen receptors. Blood 93:1809-1816 (1999).
22. Kent, D. G., Dykstra, B. J., Cheyne, J., Ma, E. & Eaves, C. J. Steel factor coordinately regulates the molecular signature and biologic function of hematopoietic stem cells. Blood 112, 560-567 (2008).
23. Audet, J., Miller, C. L., Rose-John, S., Piret, J. M. & Eaves, C. J. Distinct role of gp130 activation in prooting self-renewal divisions by mitogenically stimulated murine hematopoietic stem cells. Proc. Natl. Acad. Sci. USA 98, 1747-1762 (2001).
24. Mangi, M. H. & Newland, A. C. Interleukin-3 in hematology and oncology: current state of knowledge and future directions. Cytokines Cell Mol. Ther. 5, 87-95 (1999).
25. Nie, Y., Han, Y. & Zou, Y. CXCR4 is required fo rhte quiescence of primitive hematopoietic cells. J. Exp. Med. 205, 777-783 (2008).
26. Meyerson E U, MacLennan G, Husel W, Tse W, Lazarus H M, Kaplan D. D cyclins in CD5+B-cell lymphocproliferative disorders. Cyclin D1 and cyclin D2 identfy diagnostic gropus and cyclin D1 correlates with ZAP-70 expression in chronic lymphocytic leukemia. Am J Clin Pathol 2006; 125: 241-250.
27, Kaplan D. 2003. Enzymatic amplification staining for cell surface antigens. In Current protocols in cytometry. J. P. Robinson, editor. New York, N.Y.: Wiley, 2003. 6.14.1-6.14.11.
28. Kaplan D, Meyerson H, Husel W, Lewandowska K, MacLennan G. D cyclins in lymphocytes. Cytometry 2005; 63A:1-9.
29. Kaplan D, Smith D, Meyerson H, Pecora N, Lewandowska, K. CD5 expression by B lymphocytes and its regulation upon Epstein-Barr Virus transformation. Proc Natl Acad Sci USA 2001; 98:13850-13853.
30. Kaplan, D, Meyerson H J, Li X, Drasny C, Liu F, Costaldi M, Barr P, Lazarus H M Correlation between ZAP-70, phospho-ZAP-70, and phospho-Syk expression in leukemic cells from patients with CLL. Cytometry B 78:115-122, 2010.
31. Kadereit S, Deeds L S, Haynesworth S E, Koc O N, Kozik M M, Szekely E, Daum-Woods K, Goetchius G W, Fu P, Welniak L A, Murphy W J, Laughlin M J. Expansion of LTC-ICs and maintenance of p21 and BCL-2 expression in cord blood CD34(+)/CD38(−) early progenitors cultured over human MSCs as a feeder layer. Stem Cells 2002; 20:573-582.
32. Schiedlmeier B, Santos A C, Riberio A, Moncaut N, Lesinshi D, Auer H, Kornacker K, Ostertag W, Baum C, Mallo M, Klump H.2007. HOXB4's road map to stem cell expansion. Proc Natl Acad Sci USA 104:16952-16957.
33. Haddad R, Pflumio F, Vigon I, Visentin G, Auvray C, Fichleson S, Amsellem S. 2008. The HOXB4 homeoprotein differentially promotes ex vivo expansion of early human lymphoid progenitors. Stem Cells 26:312-322.
34. Tang Y, Chen J, Young N S. 2009. Expansion of haematopoietic stem cells from normal donors and bone marrow failure patients by recombinant hoxb4. Br J Haemotol 144:603-612.
35. Watts K L, Delaney C, Humphries R K, Bernstein I D, Kiem H-P. 2010. Combination of HOXB4 and Delta-1 ligand improves expansion of cord blood cells. Blood 116:5859-5866.
36. Wilson A, Laurenti E, Trumpp A. 2009. Balancing dormant and self-renewing hematopoietic stem cells. Genes Dev 19:1-8.
37. Wilson A, Laurenti E, Oser G, van der Wath R C, Blanco-Bose W E, Jaworski M, Offner S, Dunant C F, Eshkind L, Bockamp, Lio P, MacDonald H R, Trumpp A. 2008. Hematopoietic stem cells reversibly switch from dormancy to self-renewal during homeostasis and repair. Cell 135:1118-1129.
38. Foudi A, Hochedlinger K, Van Buren D, Schindler J W, Jaenisch R, Carey V, Hock H.2009. Analysis of histone 2B-GFP retention reveals slowly cycling hematopoietic stem cells. Nat Biotechnol 27:84-90.
39. Ruiz A, Salvo V A, Ruiz L A, Baez P, Garcia M, Flores I. 2010. Basal and steroid hormone-regulated expression of CXCR4 in human endometrium and endometriosis. Reprod Sci 17:894-903.
40. Fukumoto S, Hsieh C, Maemura K, Layne M D, Yet S, Lee, K, Matsui T, Rosenzwieg A, Taylor W G, Rubin J S, Perrella M A, Lee M. 2001. Akt participation in the Wnt signaling pathway through Dishevelled. J Biol Chem 276:17479-17483.
41. Yamazaki S, Iwama A, Takayanagi S, Morita Y, Eto K, Ema H, Nakauchi H.2006. Cytokine signals modulated via lipid rafts mimic niche signals and induce hibernation in hematopoietic stem cells. EMBO J. 25:3515-3523.
42. Wilson A, Laurenti E, Trumpp A. 2009. Balancing dormant and self-renewing hematopoietic stem cells. Curr Op Genet Develop 19:1-8.

Various publications, patent applications and patents are cited herein, the disclosures of which are incorporated by reference in their entireties.

While the invention has been explained in relation to various embodiments, it is to be understood that various modifications thereof will become apparent to those skilled in the art upon reading this specification. Therefore, it is to be understood that the invention provided herein is intended to cover such modifications as may fall within the scope of the appended claims.

1. A method for assessing the capacity of a sample to therapeutically effect hematopoietic reconstitution in a subject, the method comprising: assessing co-expression of CD34 and HoxB4 in individual cells in the sample and determining the number of cells co-expressing CD34 and levels of HoxB4 that effect therapeutic levels of self-renewing proliferation and the number of cells co-expressing CD34 and levels of HoxB4 that effect therapeutic levels of differentiation into hematopoietic cells.

2. A method to therapeutically effect hematopoietic reconstitution in a subject, the method comprising administering to a subject an agent that increases the expression of HoxB4 in CD34⁺ cells in the subject so as to provide a therapeutically effective amount of cells co-expressing CD34 and levels of HoxB4 that effect therapeutic levels of self-renewing proliferation.

3. A method to therapeutically effect hematopoietic reconstitution in a subject, the method comprising administering an agent that decreases the levels of HoxB4 in CD34⁺ cells in a subject so as to produce a therapeutically effective amount of cells co-expressing CD34 and levels of HoxB4 that effect therapeutic levels of differentiation into hematopoietic cells.

4. The method of claim 1 wherein the sample is from bone marrow.

5. The method of claim 1 wherein the sample is obtained from blood.

6. The method of claim 5 wherein the blood is umbilical cord blood.

7. The method of claim 5 wherein the blood is mobilized peripheral blood.

8. The method of claim 1 wherein the subject has a hematopoietic deficiency or malignancy.

9. The method of claim 8 wherein the subject has undergone myeloablation.

10. The method of claim 1 wherein the assessing is performed by flow cytometry.

11. A method to prepare a subject to donate blood for hematopoietic-reconstituting cell (HRC) transplantation, the method comprising obtaining a blood sample containing hematopoietic cells from the subject; determining the CD34 and HoxB4 expression levels in individual cells from the blood sample; and administering to the subject a mobilizing agent when the blood sample does not contain a therapeutically effective amount of either (a) CD34⁺ cells expressing levels of HoxB4 for self-renewal of hematopoietic-reconstituting cells or (b) CD34⁺ cells expressing levels of HoxB4 for differentiation of hematopoietic-reconstituting cells into hematopoietic cells.

12. The method of claim 11 wherein the blood sample is obtained from mobilized peripheral blood.

13. The method of claim 11 further comprising the step of administering a mobilizing agent to the subject prior to the step of obtaining a blood sample.

14. A method for transplanting hematopoietic-reconstituting cells in a subject in need thereof, the method comprising administering to the subject nucleated blood cells comprising a therapeutically effective amount of (a) CD34⁺ cells expressing levels of HoxB4 for self-renewal of hematopoietic-reconstituting cells and (b) CD34⁺ cells expressing levels of HoxB4 for differentiation of hematopoietic-reconstituting cells into hematopoietic cells.

15. The method of claim 14 wherein the CD34⁺ cells expressing the two levels of HoxB4 are isolated.

16. The method of claim 14 wherein the subject has a disorder treatable by hematopoietic stem cell transplantation.

17. The method of claim 16 wherein the disorder is a hematopoietic deficiency or malignancy.

18. The method of claim 14 wherein the CD34⁺ cells expressing the two levels of HoxB4 are in combination before administration to the subject.

19. The method of claim 14 wherein the CD34⁺ cells expressing the two levels of HoxB4 are administered sequentially.

20. The method of claim 14 wherein the CD34⁺ cells expressing the two levels of HoxB4 are administered separately.

21. A composition comprising a therapeutically effective amount of (a) CD34⁺ cells expressing levels of HoxB4 for self-renewal of hematopoietic-reconstituting cells and/or (b) CD34⁺ cells expressing levels of HoxB4 for differentiation of hematopoietic-reconstituting cells into hematopoietic cells.

22. The composition of claim 21 wherein the cells (a) and (b) are greater than 10% pure.
